(19) United States
(12) Patent Application Publication
(10) Pub. No.: US 2018/0160752 A1
(43) Pub. Date: Jun. 14, 2018

(54) POCKET POUCH

(71) Applicant: Patricia Elizabeth Morgan, Upper Marlboro, MD (US)

(72) Inventor: Patricia Elizabeth Morgan, Upper Marlboro, MD (US)

(21) Appl. No.: 15/890,316

(22) Filed: Feb. 6, 2018

Related U.S. Application Data
(63) Continuation-in-part of application No. 15/097,549, filed on Apr. 13, 2016.

(51) Int. Cl. A41D 27/20; A41B 13/10; A41B 13/04; A41B 9/12; A61F 13/49

(52) U.S. Cl. CPC A41D 27/208; A41B 13/103; A41B 13/04; A61F 2013/8476; A41D 27/201; A61F 13/49; A41D 2400/48; A41B 9/12

(57) ABSTRACT

Provided herein is a pocket pouch comprising one or more absorbency layers; a moisture free liner; and one or more resealable strips. Also provided herein are methods for attaching the pocket pouch to a cloth or disposable diaper, training pant, disposable bladder leak underwear, sanitary napkins, and/or disposable bib.

### CROSS-REFERENCES TO RELATED APPLICATIONS

This application claims priority to, and incorporates by reference herein in its entirety, pending U.S. patent application Ser. No. 15/097,549, filed Apr. 13, 2016; which claimed priority to U.S. Provisional Patent Application Ser. No. 62/282,277, which was filed on Jul. 30, 2015.

### BACKGROUND

The invention described herein relates to an item of convenience for a variety of individuals and service providers, including but not limited to: parents; daycare providers; healthcare providers; hospitals; in-home care; assisted living; individuals who use sanitary napkins and/or disposable bladder leak underwear; restaurants or other establishments that offer bibs to their patrons (e.g., Red Lobster®, Olive Garden®); and companies that provide clothing for animals and humans.

Parents, providers, and/or servers in these industries are required to gather multiple items at a time, requiring the use of both hands and loss of time. For example, when performing a diaper change or packing a diaper bag, the provider has to gather the diapers, petroleum jelly, wipes and a disposable bag. Pulling all of this together becomes inconvenient and providers on the go often forget to grab one or more necessities. The invention provided herein alleviates these issues

### SUMMARY

Provided herein is a pocket pouch, which includes: moisture free lining, resealable strips (e.g., strips that include an adhesive; Velcro® or other means to secure the contents); and the contents required for the particular task being accomplished. For example, in some non-limiting embodiments of the invention, a pocket pouch for changing a diaper could contain: individual wipes; a 1-5 oz container of petroleum jelly; a 1-5 oz container of lotion; a 1-5 oz container of baby powder; and a disposable bag all attached to the disposable diaper.

In some embodiments, the pocket pouch and its contents are releasably coupled to a cloth diaper; training pant; or disposable bib. In various embodiments, the pocket pouch contains two or more of the individual contents to allow for multiple changes.

In specific embodiments, the pocket pouch for changing a diaper contains: 2 individual wipes; a 3″×3″ package wipe size 8″×6″; 5 oz. container of petroleum jelly; 5 oz container of lotion; 5 oz. container of container of baby powder; and a disposable bag all releasably coupled to the disposable diaper.

In various embodiments, the pocket pouch varies in shape, size, colors, and type of material (recyclables) depending on the embodiment.

In some embodiments, the contents of the pocket pouch are hypoallergenic; fragrance and alcohol free; sensitive; ecologically friendly; and easy to travel with.

Accordingly, the invention described herein affords greater convenience to parents; providers; servers and other individuals to have at hand everything needed in a single easy to use pouch. This allows for easy travel, no worries about running out of sanitary condiments, and the eco-friendly safe disposal of soiled items in private or public environments.

### BRIEF DESCRIPTION OF THE DRAWINGS

A wide variety of potential practical and useful embodiments will be more readily understood through the following detailed description of certain exemplary embodiments, with reference to the accompanying exemplary drawings in which:

FIG. 1 and FIG. 2 illustrate the pocket pouch attached to the interior/back of disposable diaper with a moisture free lining and resealable strips, according to these embodiments of the invention;

FIG. 3 illustrates an embodiment of the pocket pouch attached to the interior/back of training pant with a moisture free lining and resealable strips, according to one embodiment of the invention.

FIG. 4 illustrates an embodiment of the pocket pouch attached to the disposable bib through the use of adhesive shoulder straps with a moisture free lining and resealable strips, according to one embodiment of the invention.

### DETAILED DESCRIPTION

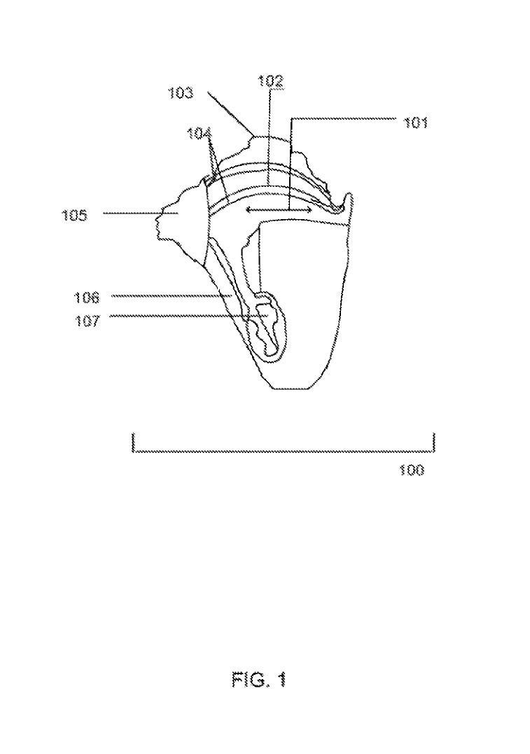

In accordance with one aspect of the invention, FIG. 1 illustrates an embodiment of the pocket pouch releasably coupled and/or releasably coupleable to the interior/back of disposable diaper. In this embodiment, the pocket pouch (100) includes: hypoallergenic absorbency layers with a full absorbency layer attached to leak guards (101); a moisture free liner 3½ inches deep (102); a stretch waistband (103); resealable strips (104); a soft stretch side panel(s) (105); soft, breathable stretch leak guards (106); and a soft inner liner with vitamin E and aloe (107) that integrates from the front of the diaper to the back of the diaper. In other embodiments, the pocket pouch is made as an individual pouch. Depending on the use and preference of the user, embodiments of the pocket pouch vary in shape, size, color and type of material.

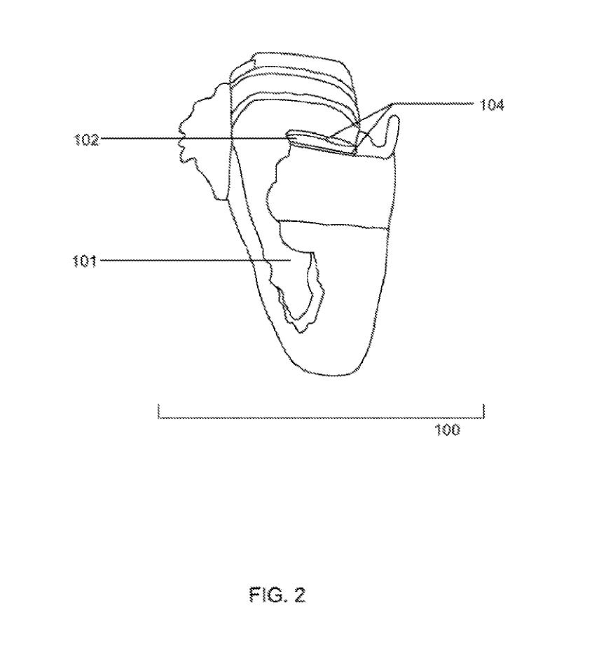

In accordance with one aspect of the invention, FIG. 2 illustrates an all-in-one disposable diaper. In this embodiment, the pocket pouch (100) coupled to a disposable diaper, which pocket pouch (100) comprises a right pocket opening to a moisture free lining (102); absorbency layers 101, which can be hypoallergenic; and resealable strips (104). In this illustrative embodiment, the pocket pouch is located on the Interior/Front of the disposable diaper. There are two absorbency layers (not shown) integrated in the diaper, from the interior/back to the interior/front of the diaper with a moisture free lining that stows the sanitary articles and resealable strips to secure the contents and to form the pocket pouch in the front of the diaper. The resealable strips can be made from made of adhesive, Velcro®, or any other suitable fastening material/means known in the art.

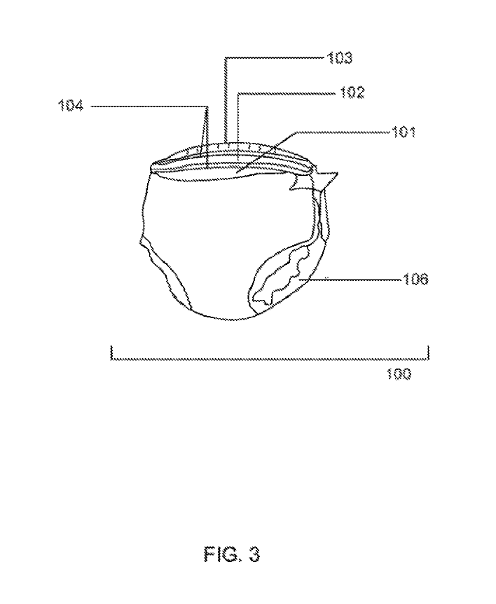

In accordance with another aspect of the invention, FIG. 3 illustrates an embodiment of the pocket pouch Pocket Pouch attached to the interior/back of training pant. This embodiment includes the pocket pouch with absorbency layers (101) a moisture free lining (102); resealable strips (104); a soft stretch waistband (103); and a soft stretch leak guard (106).

In various embodiments, the contents of the pocket pouch for disposable diapers and training pants is two (2) 3″×3″ individually packaged baby wipes (wipe size 8″×6″); one (1) 1-5 oz pack petroleum jelly; one (1) 3″×3″ (bag size 8″×6″ sealable (black or dark color) disposable bag (fold size) 3″×3″ (bag size 8″×6″), The no strings attached disposable bibs two (2) 3″×3″ individually packaged hand wipes (wipe size 8″×6″), (1) 1-5 oz pack lotion and disposable leak underwear, sanitary napkins, etc.; two (2) vaginal wipes and a disposable bag. The contents of the pocket pouch are hypoallergenic, alcohol and fragrance free, sensitive, eco-friendly, and easy to travel.

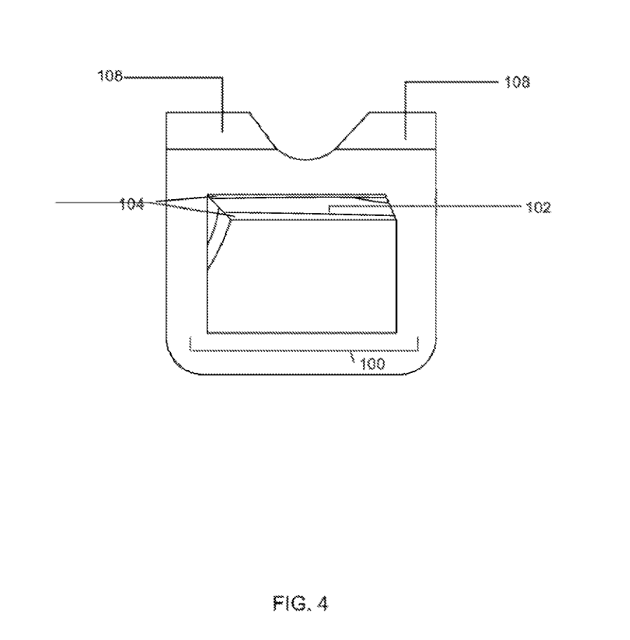

In accordance with still another aspect of the invention, FIG. 4 illustrates an embodiment of the pocket pouch attached through the use of adhesive straps (108). This embodiment includes the pocket pouch (100); resealable strips (104); moisture free lining (102); and adhesive straps (108).

Provided in the illustrated embodiments, the pocket pouch that holds or stows the contents is secured by resealable strips, tape, Velcro® or other attachment means know in the art. It should be understood that the location of the pocket pouch has been provided for illustrative purposes.

In further embodiment, the bib is attached on the human at the shoulders using adhesive, Velcro® or other. The pocket pouch also includes, resealable strips (to open and close the pouch) to secure stowage in a moisture free lining. The pocket pouch can also be embodied on the exterior front and back of the diaper, training pant, or other.

In further embodiment, said pocket pouch embodied in the diaper, training pant, bib or other provides, stowage for everything needed for a diaper change etc. The product described herein makes life easier, not having to gather each item, worry about running out of wipes, petroleum jelly etc., but more specifically the pocket pouch allows an individual, peace of mind, at home, work, in public, or private, and allows for easy travel.

Certain exemplary embodiments provide a Storage (space/area) between two absorbency layers, housed between the top-sheet and back-sheet in the front of a Biodegradable Disposable Diaper (infant to adults sizes). FIG. 2 illustrates the biodegradable diaper with the storage (space/area) in the front and FIG. 3 illustrates storage (space/area) in the front of the Training Pant.

The width (approximately 2 to 8 inches) and the length will be (approximately 2 to 8 inches) depending on the product embodiment.

The storage (space/area) can comprise a color plastic bag with resealable strips which will serve two purposes, (1) house the exact quantity of everything needed for a single diaper change, and (2) to stow and safely dispose of the soiled items, in public or private, can't see or smell soiled items, making it eco-friendly.

FIG. 4 illustrates a no strings attached disposable bib with a storage (space/area). Certain exemplary embodiments vary in colors, sizes, lengths, widths, materials and FIGS. 3 and 3a illustrates where the Bib attaches to the shoulder with adhesive backing that safely attaches on/to the shoulders (no fear of choking, back strain). Storage (space/area) stows (2) disposable facial/hand wipes.

All contents stowed/contained in the Storage (space/area) provided in illustrated embodiments described in this application or future products can be alcohol and/or fragrance free, sensitive, eco-friendly and/or adaptable for easy travel.

Certain exemplary embodiments provide a system comprising a pocket pouch. The pocket pouch is coupled, releasably coupled, and/or releasably coupleable to a sanitation item. The pocket pouch can be coupleable to an inner surface or an outer surface of the sanitation item. The sanitation item can be a diaper, training pant, bladder leak underwear, a sanitary napkin, and/or a bib.

The pocket pouch can comprise:
one or more absorbency layers;
a moisture-free liner; and/or
one or more resealable strips, etc.

The pocket pouch can be attached to an interior surface of the diaper. The resealable strips can comprise adhesive, tape, and/or Velcro, etc.

The pocket pouch can be constructed to hold individually wrapped wipes, disposable bags, a sanitation item, a 5 ounce container of petroleum jelly, and/or a 5 ounce container of lotion, etc.

### Note

Still other substantially and specifically practical and useful embodiments will become readily apparent to those skilled in this art from reading the above-recited and/or herein-included detailed description and/or drawings of certain exemplary embodiments. It should be understood that numerous variations, modifications, and additional embodiments are possible, and accordingly, all such variations, modifications, and embodiments are to be regarded as being within the scope of this application.

Thus, regardless of the content of any portion (e.g., title, field, background, summary, description, abstract, drawing figure, etc.) of this application, unless clearly specified to the contrary, such as via explicit definition, assertion, or argument, with respect to any claim, whether of this application and/or any claim of any application claiming priority hereto, and whether originally presented or otherwise:
there is no requirement for the inclusion of any particular described or illustrated characteristic, function, activity, or element, any particular sequence of activities, or any particular interrelationship of elements;
no characteristic, function, activity, or element is “essential”;
any elements can be integrated, segregated, and/or duplicated;
any activity can be repeated, any activity can be performed by multiple entities, and/or any activity can be performed in multiple jurisdictions; and
any activity or element can be specifically excluded, the sequence of activities can vary, and/or the interrelationship of elements can vary.

Moreover, when any number or range is described herein, unless clearly stated otherwise, that number or range is approximate. When any range is described herein, unless clearly stated otherwise, that range includes all values therein and all subranges therein. For example, if a range of 1 to 10 is described, that range includes all values therebetween, such as for example, 1.1, 2.5, 3.335, 5, 6.179, 8.9999, etc., and includes all subranges therebetween, such as for example, 1 to 3.65, 2.8 to 8.14, 1.93 to 9, etc.

When any claim element is followed by a drawing element number, that drawing element number is exemplary and non-limiting on claim scope. No claim of this application is intended to invoke paragraph six of 35 USC 112 unless the precise phrase “means for” is followed by a gerund.

Any information in any material (e.g., a United States patent, United States patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such material is specifically not incorporated by reference herein.

Accordingly, every portion (e.g., title, field, background, summary, description, abstract, drawing figure, etc.) of this application, other than the claims themselves, is to be regarded as illustrative in nature, and not as restrictive, and the scope of subject matter protected by any patent that issues based on this application is defined only by the claims of that patent.

What is claimed is:

1. A system comprising:
a pocket pouch releasably coupled to a diaper, the pocket pouch comprising:
one or more absorbency layers;
a moisture-free liner; and
one or more resealable strips;
wherein the pocket pouch is attached to an interior surface of the diaper.

2. The system of claim 1, wherein:
the resealable strips comprise adhesive.

3. The system of claim 1, wherein:
the resealable strips comprise tape.

4. The system of claim 1, wherein:
the resealable strips comprise Velcro.

5. The system of claim 1, wherein:
the pocket pouch is constructed to hold individually wrapped wipes.

6. The system of claim 1, wherein:
the pocket pouch is constructed to hold disposable bags.

7. The system of claim 1, wherein:
the pocket pouch is constructed to hold a sanitation item.

8. The system of claim 1, wherein:
the pocket pouch is constructed to hold a 5 ounce container of petroleum jelly.

9. The system of claim 1, wherein:
the pocket pouch is constructed to hold a 5 ounce container of lotion.

10. A system comprising:
a pocket pouch releasably coupleable to a sanitation item, the pocket pouch comprising:
one or more absorbency layers;
a moisture-free liner; and
one or more resealable strips;
wherein the pocket pouch is attached to an interior surface of the sanitation item.

11. The system of claim 10, wherein:
the sanitation item is a training pant.

12. The system of claim 10, wherein:
the sanitation item is bladder leak underwear.

13. The system of claim 10, wherein:
the sanitation item is a sanitary napkin.

14. The system of claim 10, wherein:
the sanitation item is a bib.
